(19) United States
(12) Patent Application Publication
(10) Pub. No.: US 2016/0339300 A1
(43) Pub. Date: Nov. 24, 2016

(54) CONTROLLING USER DEVICES BASED ON BIOMETRIC READINGS

(71) Applicant: EBAY INC., San Jose, CA (US)

(72) Inventor: Michael Charles Todasco, Santa Clara, CA (US)

(21) Appl. No.: 14/718,496

(22) Filed: May 21, 2015

(51) Int. Cl. A63B 24/00; H04W 4/00; G05B 15/02; A61B 5/11; A61B 5/00

(52) U.S. Cl. CPC A63B 24/0087; A61B 5/1118; A61B 5/4866; A63B 24/0062; G05B 15/02; H04W 4/008; A63B 2024/0065; A63B 2024/0068; A63B 2024/0093

(57) ABSTRACT

A system and method for controlling one or more devices in the vicinity of a user based on biometric of the user and a target user state. The system and method determines the target state, which is characterized by biometric measurements, through either a user entered command or a prediction based on historic user entered commands. Using biometric readings of the user as feedback, the one or more devices are controlled to cause the biometrics of the user to change towards the target state.

### BACKGROUND

1. Field of the Disclosure

The present disclosure generally relates to Internet of Things. More specifically, controlling devices using biometric readings of a user.

2. Related Art

In recent times, many portable devices have been developed for monitoring and displaying the biometrics of a user. For example, pedometers have been created for users to estimate distance traveled, calories burned, and mapping travel routes. Similarly, there are devices for measuring, tracking, and displaying body mass index (BMI), body fat percentage, heartbeats, blood pressure, blood sugar levels, perspiration, muscle activity, brainwaves, temperature, calories burned, distance traveled, number of steps taken, and/or the like. However, all of these devices are designed such that a user reacts to the device rather than the device reacting to the user. For example, a calorie tracker informs a user about the amount of calories the user has eaten and/or burned, but the user would have to act upon that information, such as exercising more or limiting calories to receive a benefit. Another example may be a muscle monitor that informs the user which muscles have been active so that the user can decide which muscles need exercise.

While these devices have enabled users to monitor their progress on their health goals and gain new insights into their own body metrics, it is still up to the user to act upon that data. Furthermore, as more and more data is tracked and provided to a user, the user may be overloaded with all the information. A user may find all the different numbers and metrics provided by several devices confusing. Furthermore, a user may find it difficult to decide on a best course of action when the biometric information conflict. For example, a user may have a blood pressure monitor indicating that the user should rest, but the user may also have a muscle activity monitor indicating that the user should exercise their legs to maintain their fitness goals. The user may also be tracking additional information such as pedometer readings, blood sugar readings, and/or the like. In such a scenario, the user would have to actively decide on a regular basis what goals are more important and how to best respond to the biometric information.

It would be beneficial if a user could input or set fitness goals into a system and have the system automatically manipulate the environment based on biometric measurements to achieve those goals. Furthermore, it would be beneficial if a system could manipulate an environment to achieve other goals, such as maintaining and/or reaching an emotional state, maintaining focus, relaxing, falling asleep, maintaining a routine, staying awake, and/or the like. It would also be desirable if the system could automatically determine user goals rather than have a user actively input or set a goal.

### BRIEF DESCRIPTION OF THE FIGURES

FIG. 1 is a block diagram of an exemplary system for controlling user stimulus based on biometric readings.

FIG. 2 is a block diagram of an exemplary computer system suitable for implementing one or more devices of the computing system in FIG. 1 and the embodiments in this disclosure.

FIG. 3 illustrates a user with several devices implementing an exemplary system for controlling user stimulus based on biometric readings.

FIG. 4. Is a flow diagram illustrating an exemplary method of manipulating a user environment based on biometric readings.

FIG. 5 is a flow diagram illustrating an exemplary method for adjusting a user environment based on biometric signals for maintaining a user routine.

FIG. 6 is a flow diagram illustrating an exemplary method for tailoring a fitness activity based on biometric readings.

FIG. 7 is a flow diagram illustrating an exemplary method for automatically determining a target user state.

FIG. 8 is a flow diagram illustrating an exemplary method for modeling a state of the user and how a user will respond to system controlled stimuli.

Embodiments of the present disclosure and their advantages are best understood by referring to the detailed description that follows. It should be appreciated that like reference numerals are used to identify like elements illustrated in one or more of the figures, wherein showings therein are for purposes of illustrating embodiments of the present disclosure and not for purposes of limiting the same.

### DETAILED DESCRIPTION

In the following description, specific details are set forth describing some embodiments consistent with the present disclosure. It will be apparent, however, to one skilled in the art that some embodiments may be practiced without some or all of these specific details. The specific embodiments disclosed herein are meant to be illustrative but not limiting. One skilled in the art may realize other elements that, although not specifically described here, are within the scope and the spirit of this disclosure. In addition, to avoid unnecessary repetition, one or more features shown and described in association with one embodiment may be incorporated into other embodiments unless specifically described otherwise or if the one or more features would make an embodiment non-functional.

Some embodiments disclosed herein include a system that gathers biometric measurements from one or more devices and uses the biometric measurements as feedback to adjust a state of the user by manipulating electronic devices in the user environment. For example, a system may gather heartbeat measurements from a heartbeat monitor, and in response, cause a media player to play music coinciding with causing changes to the heartbeat of a person. The system may monitor changes in the heartbeat measurements from the heartbeat monitor as feedback and cause a media player to change songs, volume levels, equalizer settings and/or the like accordingly to achieve a desired heartbeat. The same may be applied to other biometric measurements (e.g. brainwaves, blood pressure, etc.) and other electronic devices (e.g. light dimming levels, television settings, aroma therapy devices, heating ventilation and air conditioning (HVAC) systems, display settings, and/or the like). In some examples, the system may use a combination of multiple biometric measurements as feedback for adjusting the state of the user and/or recognizing a state of the user.

In some examples, the system may aid a user to achieve and/or maintain a focused state, a sleep state, an awake state, calm state, homeostasis stats, and/or the like. In some examples, the system may aid the user in reducing stress, increasing comfort and/or the like. The system may use one or more training sets, which may be created and/or changed over time, to determine a state of the user and manipulate one or more devices in the environment of the user to adjust a current user state to a target user state. In some examples, the system may use one or more training sets to determine how to manipulate the one or more devices in a manner that will cause the current user state to move towards a target user state. In some examples, one or more of the training sets may be created over time through the user using the system. In some examples, there may be an initial training set which is adapted to the user over time.

In some embodiments, the system may aid a user in maintaining a routine and/or schedule. For example, a user may use the system to help maintain a sleeping, wakeup, and/or exercise schedule by controlling devices around the user to put the user in a state ready for sleeping, wakeup, and/or exercise during certain times of the day. In some examples, the system may attempt to change the user state gradually to prevent a jarring experience. For example, the system may have a predetermined training set for emotional state and a predetermined training set for user reactions to system controlled devices. The system may update the training set with new data points based on actual emotional states and/or reactions of the user.

In some embodiments, the system may use one or more biometric measurements to tailor or make adjustments in real-time to an exercise routine for the user. The system may, based on biometric readings over a period of time, change the intensity settings for an exercise machine and/or program. In some embodiments, the system may receive biometric measurements and adjust the exercise intensity or pace in real time. In some embodiments, the system may use a combination of biometric readings over a period of time and in real time to adjust the intensity of an exercise machine and/or program. For example, the system may receive pedometer readings and/or calorie intake/burn readings and adjust an exercise program for the user based on a combination of those readings and possibly other biometric readings. In some examples, the system may suggest or setup an exercise program for muscles that have been neglected and/or avoid recently exercised muscles.

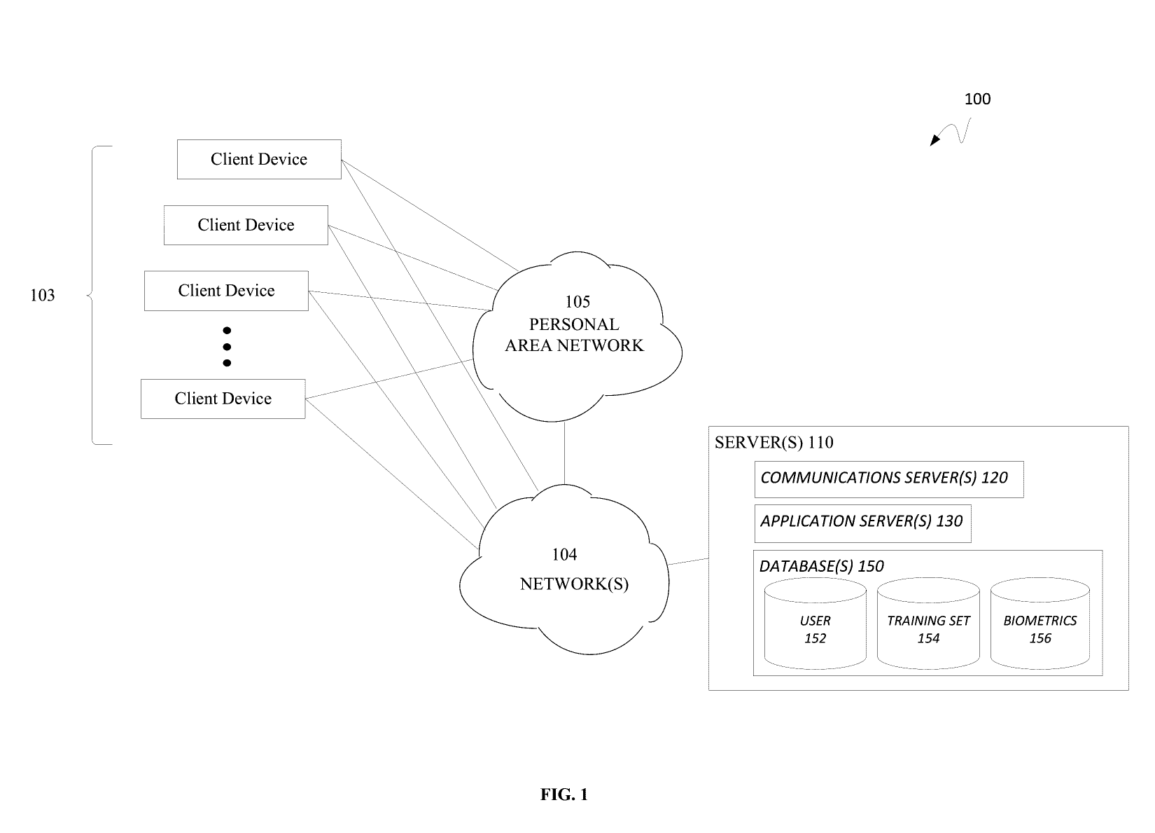

FIG. 1 illustrates an exemplary system 100 for controlling user stimulus based on biometric readings. As shown, a computing system 100 may comprise or implement a plurality of computer devices, servers, and/or software components that operate to perform various methodologies in accordance with the described embodiments. Exemplary servers may include, for example, stand-alone and enterprise-class servers operating a server operating system (OS) such as a MICROSOFT® OS, a UNIX® OS, a LINUX® OS, or other suitable server-based OS. It may be appreciated that the devices illustrated in FIG. 1 may be deployed in other ways and that the operations performed and/or the services provided by such devices may be combined, distributed, and/or separated for a given implementation and may be performed by a greater number or fewer number of devices. One or more devices may be operated and/or maintained by the same or different entities.

Computing system 100 may include, among various devices, servers, databases and other elements, one or more client devices 103. Client devices 103 may be categorized into three categories such as sensor devices, user stimulus devices, and computing devices. Sensor devices as used herein are devices with a sensor capable of determining a user biometric. Sensor devices may include, but are not limited to, devices with accelerometers, gyroscopes, electroencephalography (EEG) monitors, magnetoencephalography (MEG) monitors, electromyography (EMG) monitors, brainwave scanners, heat scanners, bioelectrical impedance (BIA) monitors, pressure sensors, pedometers, blood pressure monitors, pulse monitor, breathing monitor, breathalyzer, perspiration monitor, muscle activity sensors (e.g. devices for detecting masseter motion), motion sensors, microphones, and/or the like. Some sensor devices may have a singular biometric sensor while other devices may contain multiple biometric sensors.

User stimulus devices as used herein are devices capable of providing one or more stimulants to a user. Stimulus devices may include, but are not limited to, lighting devices, devices capable of haptic feedback, televisions, media devices (e.g. iPods, CD players, radios, speakers, DVD players, etc.), aromatherapy devices, exercise equipment, heat pads, HVAC systems, thermostats, alarms, moving desks, and/or the like. In some embodiments, a single device may be in multiple categories. For example, a smart phone is a computing device which likely has some sort of biometric sensor and haptic feedback. Thus, the smart phone is a computing device, sensor device, and a user stimulus device.

Some devices which client devices 103 may include, but are not limited to, are devices such as laptops, mobile computing devices, tablets, personal computers, wearable electronic devices, cellular telephones, smart phones, smart televisions (TVs), digital media players, virtual reality headsets, augmented reality headsets, and/or the like. Client devices 103 generally may include any electronic device.

One or more of client devices 103 may provide one or more client programs, such as system programs and application programs to perform various computing and/or communications operations. Exemplary system programs may include, without limitation, an operating system (e.g., MICROSOFT® OS, UNIX® OS, LINUX® OS, Symbian OS™, Embedix OS, Binary Run-time Environment for Wireless (BREW) OS, JavaOS, a Wireless Application Protocol (WAP) OS, and others), device drivers, programming tools, utility programs, software libraries, application programming interfaces (APIs), and so forth. Exemplary application programs may include, without limitation, a web browser application, messaging applications (e.g., e-mail, IM, SMS, MMS, telephone, voicemail, VoIP, video messaging), contacts application, calendar application, electronic document application, database application, media applications (e.g., applications for music, video, television, etc.), clocks, security applications, biometric tracking applications, location-based services (LBS) applications (e.g., GPS, mapping, directions, positioning systems, geolocation, point-of-interest, locator) that may utilize hardware components such as an antenna and/or wave guide, and so forth. One or more of the client programs may display various graphical user interfaces (GUIs) to present information to and/or receive information from one or more users of client devices 103. In some embodiments, client programs may include one or more applications configured to conduct some or all of the functionalities and/or processes discussed below.

As shown, client devices 103 may be communicatively coupled via one or more networks 104 to servers 110. Server 110 may be structured, arranged, and/or configured to allow client devices 103 to establish one or more communications sessions to servers 110. Accordingly, a communications session between client devices 103 and servers 110 may involve the unidirectional and/or bidirectional exchange of information and may occur over one or more types of networks 104 depending on the mode of communication. While the embodiment of FIG. 1 illustrates a computing system 100 deployed in a client-server operating environment, it is to be understood that other suitable operating environments and/or architectures may be used in accordance with the described embodiments.

Communications between client devices 103 and the servers 110 may be sent and received over one or more networks 104 such as the Internet, a WAN, a WWAN, a WLAN, a mobile telephone network, a landline/public switched telephone network (PSTN), as well as other suitable networks. Any of a wide variety of suitable communication types between client devices 103 and system 110 may take place, as will be readily appreciated. For example, wireless communications of any suitable form may take place between client devices 103 and servers 110, such as that which often occurs in the case of mobile phones or other personal and/or mobile devices.

In some embodiments, client devices 103 may be owned, managed, or operated by a single entity, such as a person. In some embodiments, client devices 103 may form a mesh network and/or a personal area network 105. Personal area network 105 may be created using short range wireless communicators such as Bluetooth®, Bluetooth® low energy, wireless infrared communications, wireless USB, Wi-Fi and/or other wireless technologies for exchanging data over short distances. In some embodiments, one or more of client devices 103 may act as a wireless hotspot for other client devices 103 to connect to one or more networks 104 and communicate with servers 110 and/or with each other. In some embodiments, one or more of devices 103 may act as a central device for gathering and communicating control commands to other devices.

Server 110 may comprise one or more communications servers 120 to provide suitable interfaces that enable communication using various modes of communication and/or via one or more networks 104. Communications servers 120 may include a web server, an API server, and/or a messaging server to provide interfaces to one or more application servers 130. Application servers 130 of servers 110 may be structured, arranged, and/or configured to provide access to one or more applications. In some examples, application server 130 may run an application for tracking biometric data, device control services, health applications, sleep quality applications, and/or the like. Application servers 130 may include one or more applications for device authentication, account access, device detection, cross device communications, and/or the like. Application server 130 may also include one or more applications for implementing the systems and methods described herein.

In various embodiments, client devices 103 may communicate with application servers 130 of servers 110 via one or more interfaces provided by communication servers 120. It may be appreciated that servers 110 may be structured, arranged, and/or configured to communicate with various types of client devices 104 and operator devices 106.

Application servers 130, in turn, may be coupled to and capable of accessing one or more databases 150 including, but not limited to, a user database 152, a training set database 154, and/or biometrics database 156. Databases 150 generally may store and maintain various types of information for use by application servers 130 and/or other devices and may comprise or be implemented by various types of computer storage devices (e.g., servers, memory) and/or database structures (e.g., relational, object-oriented, hierarchical, dimensional, network) in accordance with the described embodiments. In some embodiments, the information held in the databases 150 may be stored on one or more of client devices 103. The data may be held in a distributed fashion and/or redundant fashion.

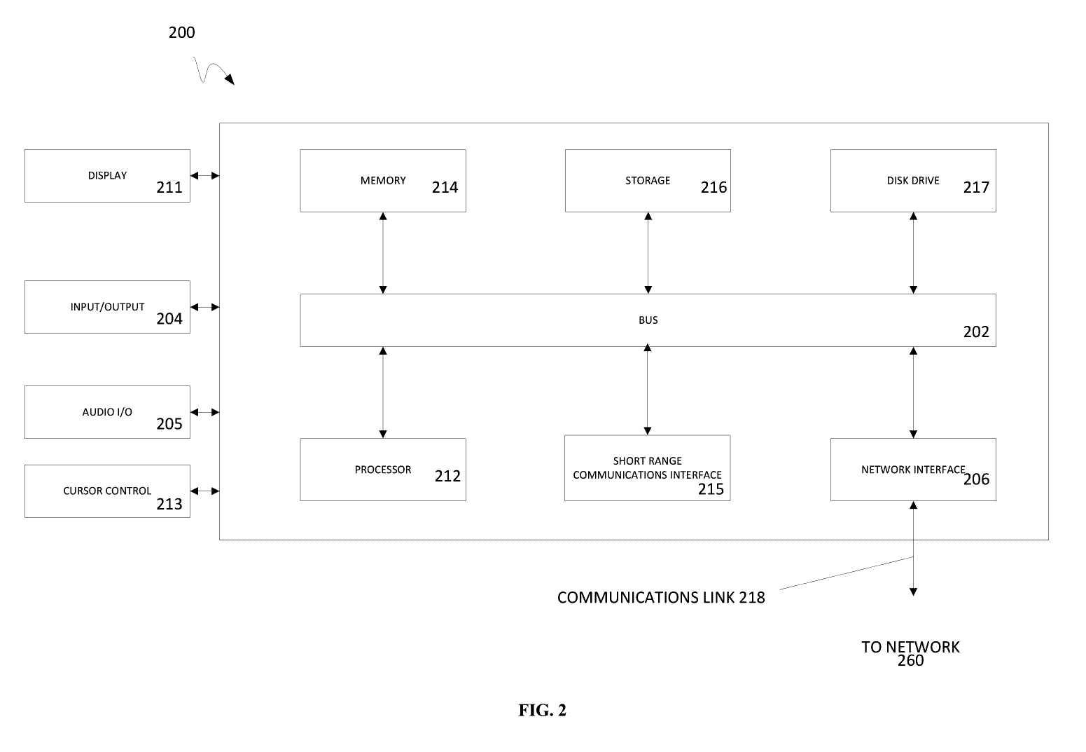

FIG. 2 illustrates an exemplary computer system 200 in block diagram format suitable for implementing one or more devices of the computing system in FIG. 1 and/or the embodiments discussed herein. In various implementations, a device that includes computer system 200 may comprise a personal computing device (e.g., a smart or mobile phone, a computing tablet, a personal computer, laptop, wearable device, PDA, Bluetooth device, etc.) that is capable of communicating with a network or another device. In some examples, computer system 200 may be a network computing device (e.g., a network server). It should be appreciated that each of the devices of the computer system in FIG. 1 may be implemented as computer system 200 in a manner as follows.

Computer system 200 may include a bus 202 or other communication mechanisms for communicating information data, signals, and information between various components of computer system 200. Computer system 200 may include an input/output (I/O) component 204 that processes a user action, such as selecting keys from a keypad/keyboard, selecting one or more buttons, links, actuatable elements, etc., and sends a corresponding signal to bus 202. Computer system 200 may also include a display 211 which may display information. In some embodiments, display 211 may double as an I/O component. For example, display 211 may be a touch screen device. In some embodiments, computer system 200 may include an audio input/output component 205. Audio input/output component 205 may be able to transmit and/or receive audio signals to and/or from the user.

Computer system 200 may include a short range communications interface 215. Short range communications interface 215 may be capable of exchanging data with other devices with short range communications interfaces. In some embodiments computer system 200 may have several short range communications interfaces using different communication protocols and may join one or more networks using short range communications interface 215.

Short range communications interface 215, in various embodiments, may include transceiver circuitry, an antenna, and/or waveguide. Short range communications interface 215 may use one or more short-range wireless communication technologies, protocols, and/or standards (e.g., WiFi, Bluetooth, Bluetooth low energy, infrared, NFC, etc.).

Short range communications interface 215, in various embodiments, may be configured to detect other devices with short range communications technology near computer system 200. Short range communications interface 215 may create a communication area for detecting other devices with short range communication capabilities. When other devices with short range communications capabilities are placed in the communication area of short range communications interface 215, short range communications interface 215 may detect the other devices and exchange data with the other devices. Short range communications interface 215 may receive identifier data packets from the other devices when in sufficiently close proximity. The identifier data packets may include one or more identifiers, which may be operating system registry entries, cookies associated with an application, identifiers associated with hardware of the other device, and/or various other appropriate identifiers.

In some embodiments, short range communications interface 215 may identify a local area network using a short range communications protocol, such as WiFi, and join the local area network. In some examples, computer system 200 may discover and/or communicate with other devices that are a part of the local area network using short range communications interface 215. In some embodiments, short range communications interface 215 may further exchange data and information with the other devices that are communicatively coupled with short range communications interface 215.

Computer system 200 may have a transceiver or network interface 206 that transmits and receives signals between computer system 200 and other devices, such as another user device, an application server, application service provider, web server, a social networking server, and/or other servers via a network. In various embodiments, this transmission may be wireless, although other transmission mediums and methods may also be suitable. A processor 212, which may be a micro-controller, digital signal processor (DSP), or other processing component, processes these various signals, such as for display on computer system 200 or transmission to other devices over a network 260 via a communication link 218. Again, communication link 218 may be a wireless communication in some embodiments. Processor 212 may also control transmission of information, such as cookies, IP addresses, and/or the like to other devices.

Components of computer system 200 may also include a system memory component 214 (e.g., RAM), a static storage component 216 (e.g., ROM), and/or a disk drive 217. Computer system 200 performs specific operations by processor 212 and other components by executing one or more sequences of instructions contained in system memory component 214. Logic may be encoded in a computer readable medium, which may refer to any medium that participates in providing instructions to processor 212 for execution. Such a medium may take many forms, including but not limited to, non-volatile media, volatile media, and/or transmission media. In various implementations, non-volatile media includes optical or magnetic disks, volatile media includes dynamic memory, such as system memory component 214, and transmission media includes coaxial cables, copper wire, and fiber optics, including wires that comprise bus 202. In one embodiment, the logic is encoded in a non-transitory machine-readable medium. In one example, transmission media may take the form of acoustic or light waves, such as those generated during radio wave, optical, and infrared data communications.

Some common forms of computer readable media include, for example, floppy disk, flexible disk, hard disk, magnetic tape, any other magnetic medium, CD-ROM, any other optical medium, punch cards, paper tape, any other physical medium with patterns of holes, RAM, PROM, EPROM, FLASH-EPROM, any other memory chip or cartridge, or any other medium from which a computer is adapted to read.

In various embodiments of the present disclosure, execution of instruction sequences to practice the present disclosure may be performed by computer system 200. In various other embodiments of the present disclosure, a plurality of computer systems 200 coupled by communication link 218 to the network (e.g., such as a LAN, WLAN, PTSN, and/or various other wired or wireless networks, including telecommunications, mobile, and cellular phone networks) may perform instruction sequences to practice the present disclosure in coordination with one another. Modules described herein may be embodied in one or more computer readable media or be in communication with one or more processors to execute or process the steps described herein. A computer system may transmit and receive messages, data, information and instructions, including one or more programs (i.e., application code) through a communication link and a communication interface. Received program code may be executed by a processor as received and/or stored in a disk drive component or some other non-volatile storage component for execution.

Where applicable, various embodiments provided by the present disclosure may be implemented using hardware, software, or combinations of hardware and software. Also, where applicable, the various hardware components and/or software components set forth herein may be combined into composite components comprising software, hardware, and/or both without departing from the spirit of the present disclosure. Where applicable, the various hardware components and/or software components set forth herein may be separated into sub-components comprising software, hardware, or both without departing from the scope of the present disclosure. In addition, where applicable, it is contemplated that software components may be implemented as hardware components and vice-versa.

Software, in accordance with the present disclosure, such as program code and/or data, may be stored on one or more computer readable media. It is also contemplated that software identified herein may be implemented using one or more computers and/or computer systems, networked and/or otherwise. Such software may be stored and/or used at one or more locations along or throughout the system, at client 103, servers 110, or both. Where applicable, the ordering of various steps described herein may be changed, combined into composite steps, and/or separated into sub-steps to provide features described herein.

The foregoing networks, systems, devices, and numerous variations thereof may be used to implement one or more services, such as the services discussed above and in more detail below.

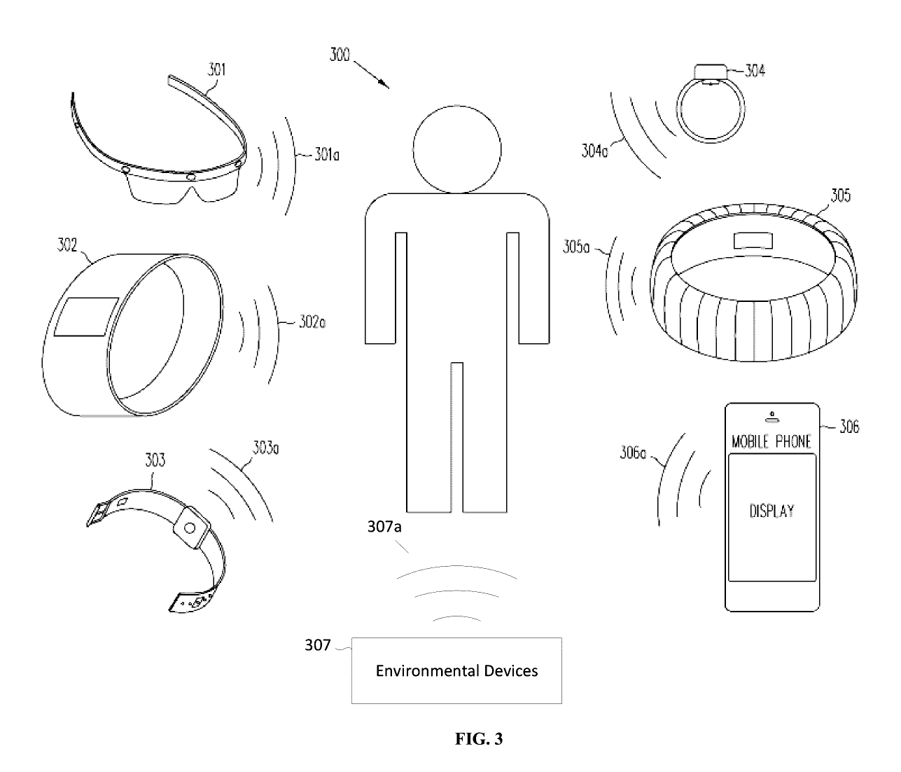

FIG. 3 illustrates a user 300 with devices 301-307 which may be a part of a system, such as system 100 of FIG. 1, for using biometric readings to determine and apply an appropriate user stimulant or sensory content (e.g., visual, vibrational, audio, smell, etc.). In some embodiments, devices 301-307 may create a personal area network using short range wireless communications 301a-307a. Short range wireless communications 301a-307a may use a single wireless communication protocol, such as Bluetooth®. In some embodiments, wireless communications 301a-307a may use multiple communication protocols, such as Bluetooth® and Wifi. Some devices may use one protocol, some devices may use another protocol, and some devices may use multiple protocols. In this manner, the personal area network may be created by multiple communication protocols. In some embodiments, the personal area network may also be connected to a wide area network or other networks, such as the internet, through one or more of devices 301-307.

In some embodiments, one or more of devices 301-307 may be configured to recognize and automatically connect with each other when in range of wireless communications 301a-307a. In some embodiments, the personal area network may implement a security measure such that there is an authentication and/or authorization process for connecting to the personal area network. In some embodiments, the security measure may use a combination of unique identifiers for the devices and an access control list for authentication. In some examples, devices 301-307 may be part of a local area network and may be able to communicate with each other through the local area network.

Devices 301-307 may include, but are not limited to, personal devices 301-306 such as eyewear 301, fitness band 302, smart watch 303, ring 304, bracelet 305, and/or smart phone 306. As technology progresses and enables more wearable objects to contain microcomputers with communication capabilities, these items may also be used in a similar manner as personal devices 301-306. Some examples may include clothing, hats, key chains, shoes, wallets, belt buckles, earrings, necklaces, cuff links, pins or brooches, tattoos, keycards, embedded medical devices, biomechanical devices, and/or the like.

Some and/or all of personal devices 301-306 may contain applications and hardware to provide a variety of services, which may include, but are not limited to, biometric monitoring, location services, and/or the like. Biometric monitoring may be conducted by one or more of personal devices 301-306 through a combination of one or more biometric sensors on the devices, such as some of the sensors discussed above in relation to system 100 of FIG. 1.

In some examples, the system may include environmental devices 307. In some examples, environmental devices 307 may be capable of providing a stimulus to the user. Some exemplary environment devices may include, but are not limited to, smart TVs, smart room lighting, electronic blinds, HVAC systems, home theatre devices, coffee makers, video game consoles, desk top computers, motor vehicles, and/or the like. The number of devices categorized under environmental devices 307 may grow as more and more everyday devices start having the capabilities to connect to a network and controlled by or used to control another device.

In some embodiments, one or more devices 301-306 may measure one or more biometrics of user 300, translate the measurements into computer readable biometric data, and communicate the biometric data to one or more of devices 301-307 and/or a remote server (not shown), such as server 110 of FIG. 1. For example, eyewear 301 may have a brainwave monitor that measures and records brainwaves, such as gamma, beta, theta, alpha, and/or delta waves. These measurements may be communicated to another device, such as a device with larger computing power and/or memory. For example, the measurements may be communicated to a master device, such as smart phone 306 or a server. Similarly, devices 302-307 may also measure biometrics of user 300, which may be different from the biometric measured by device 301, and communicate the measurements to mobile device 306. Some exemplary biometrics may include blood pressure, perspiration, temperature, heart rate, muscles activity, and/or the like. In some embodiments, a master and/or central device, such as mobile device 306, may record the measurements in a database, which may be physical memory on the master device and/or a remote database on a remote server, such as database 150 of FIG. 1.

In some embodiments, the master device may, based on user preferences, use real-time measured biometric data to control one or more devices 301-306 and/or environmental devices 307 surrounding user 300 to perform an action that introduces a stimulant to user 300. Note that as used herein, “stimulant,” “stimulus,” and the like can be anything detectable by the user to change or maintain a current state, including calming the user, as well as stimulating the user.

For example, user 300 may have entered in a user preference to smart phone 306 that user 300 would like to stay alert. Smartphone 306 may collect and analyze the biometric data to determine whether the user is falling asleep and in response cause one or more of devices 301-307 to vigorously vibrate, play a loud sound, play music, play videos, change the temperature of the room, and/or introduce other stimulants to bring user 300 to an alert state. Similarly, user 300 may have entered in a user preference that he/she would like to be calmed and/or relaxed. Smartphone 306 may collect and analyze the biometric data to recognize that user 300 is in an anxious state, and in response, cause one or more of devices 301-307 to play calming sounds and/or command one or more of the devices to introduce other stimulus that may calm the user. In some embodiments, smart phone 306 may send the biometric data to an application on one or more of device 301-307 or a remote server for analysis. The application on the remote server may implement on or more of the methods discussed in detail below for analyzing biometric data and providing an appropriate stimulus in response.

In some embodiments, the master device may, based on user preferences, use biometric data recorded over a period of time, such as twenty-four hours, to change and/or adjust a user stimulus and accordingly control one or more user devices. For example, a user may have a goal of burning a certain amount of calories. Smartphone 306 may input biometric data measured from one or more devices 301-307 into an application to determine how sedentary or active user 300 has been in the past 24 hours. In response to the user being particularly sedentary, the system may control an environment device 307 to cause the user to burn more calories. For example, the system may control an electronic table such that the table adjusts to a standing position so that the user is standing rather than sitting. In other examples, the system may adjust a programmed user exercise routine to cause the use to burn more calories and stay on track with their goals. In other examples, the system may adjust a programmed user exercise routine to be easier based on the fact that the user has been particularly active and/or over active.

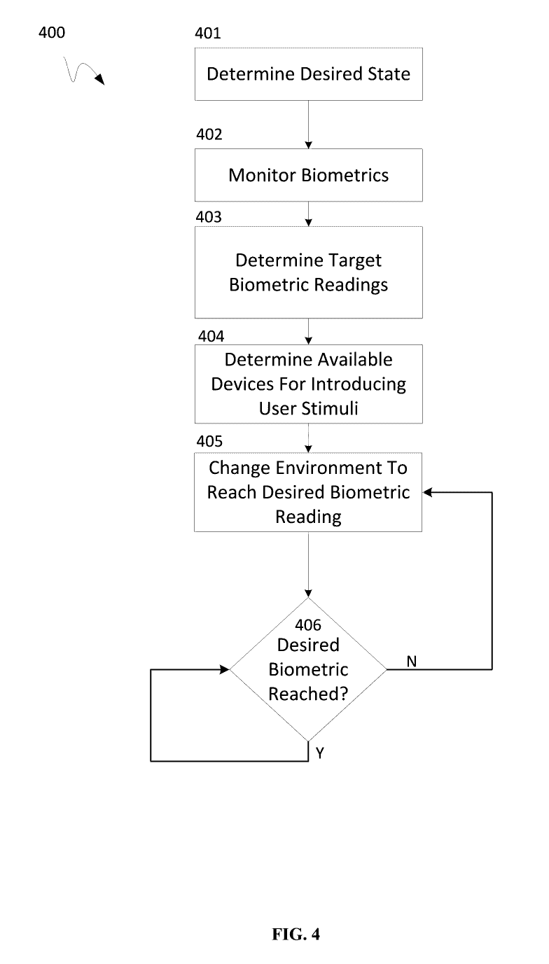

FIG. 4 illustrates a method 400 of manipulating a user environment based on biometric readings from user devices and manually entered or automated settings that may be implemented by a system, such as system 100 of FIG. 1 and/or the system described in relation to FIG. 3. According to some embodiments, method 400 may include one or more of the processes 401-406 which may be implemented, at least in part, in the form of executable code stored on a non-transitory, tangible, machine readable media that when run on one or more processors may cause the one or more processors to perform one or more of the processes 401-436.

At process 401, the system may determine a desired/target mental, behavioral, physiological, and/or emotional state of the user. In some embodiments, the system may provide the user several state options to choose from which the use may respond by selecting. In some examples, the options may be displayed on a user device, such as one or more of user devices 103 of FIG. 1 or devices 301-307 of FIG. 3. The user may respond by selecting one or more of the options using an I/O device, such as a touch screen or a mouse. Some exemplary state options may include, but are not limited to, a focused state, depressed state, alert state, relaxed state, energetic state, homeostasis, a combination of states, and/or the like. In some examples, the options may be as simple as two opposing states such as calm and excite.

In some embodiments, the system may automatically determine a desired state of the user based on historical biometric data. An example of automatically determining a desired state is discussed in more detail below.

At process 402, the system many monitor and record biometric readings of the user received from one or more devices. The biometric readings from the one or more devices may include several types, such as blood pressure, brainwaves, and others which were discussed in more detail above. In some embodiments, these may be real-time and/or near real-time readings. In some examples, the system screens for biometric reading anomalies. The system may check to ensure that the biometric readings are within normal and/or human capabilities. For example, a zero heartbeat reading is impossible for a living human. In some examples, the system may have predetermined thresholds for normal readings. In some examples, normal readings may be determined over time based on historic user readings. In some examples, a server may maintain a database of multiple user readings and use pattern recognition algorithms to determine whether a reading is anomalous. In some examples, the system may flag and/or discard abnormal readings. In some cases, the system may treat a device providing abnormal readings as if the device is inactive.

At process 403, the system may determine target biometric readings of the user. The system may create target biometric readings for each type of biometric reading received and/or for each device providing a biometric reading at process 302. In some examples, the target biometric readings may not be created for devices providing abnormal readings as discussed in process 302.

In some examples, the target biometric readings may be determined based on previously recorded biometric signatures. For example, there may be a focused state biometric signature. In some examples the signature may be made up of a combination of biometric measurements such as heartbeat measurements, brainwave measurements, body heat measurements, and/or other biometric measurements. These recorded measurements may be used as the target biometric measurements for the user if the user selected a focused state at process 301. In some examples, these biometric signatures may be created by measuring the biometrics of the user during a particular state. An example of creating a biometric signature for a particular state is discussed in more detail below. In some examples, biometric readings for a particular state may be created using empirical data.

In some examples, the system may set a target state as a predetermined quantized value away from the measurements measured at process 302. In some examples, the quantized value may be a percentage change of a measurement or a multiple of a standard deviation for a measurement, such as a 2% increase/decrease in heartbeat or one standard deviation of a heartbeat measurement. In some examples, the quantized value may be determined through empirical data and/or measurements. The system may set target measurements in this manner when the user chooses a generic desired target state at process 301, such as calm or excite.

At process 404, the system may identify available devices capable of introducing stimuli to a user. In some examples, one of more devices may be in communication with the system and communicate their capabilities to the system. In some embodiments, another device, such as a master device, may act as a communication relay between the available devices capable of introducing stimuli.

At process 405, the system may change the environment of the user by controlling one or more of the devices identified at process 404. In some examples, the system may determine one or more control models for one or more of the identified available devices for introducing user stimuli. The system may pick and introduce stimuli that will likely cause the user to change emotional, mental, and/or behavioral states so that the biometric readings of the user move towards the targeted biometric readings. The choice and method in which stimuli is introduced may be preprogrammed and/or determined from one or more training data sets. For example, the system may gradually introduce calming music from a low volume to a higher volume to reduce the heartbeat of the user, increase alpha brainwaves, reduce muscle activity in certain areas of the body, such as the jaw, and/or the like. The system may also dim lights, change the temperature of the room to a comfortable level, and/or engage an aroma therapy device. The system may play a loud alarm and/or high energy music in a jarring fashion, brighten lights, change the room temperature to an uncomfortable level, and engage different forms of aroma therapy to increase the heart rate of the user and/or beta brainwaves. Similar methods may be used to cause other biometric levels to change.

At process 306, the system may determine whether the biometric readings have reached target levels or are within acceptable ranges for the target levels. In some examples, the system may determine whether the current biometric readings are within a multiple of a standard deviation from the targeted biometric readings. When the biometric readings have reached the targeted level, the system may continue to monitor the biometric readings for deviations away from the targeted levels. When the biometric readings are not at target levels, the system may repeat process 305 for changing the environment of the user such that the biometric readings of the user align with the targeted biometric levels.

In some embodiments, the system may include a failsafe to prevent feedback loops causing the system to control one or more devices in an unreasonable manner. For example, the failsafe could prevent the system from gradually increasing the volume of a device to deafening levels in an effort to reach a targeted user state and/or biometric reading. In some examples, the system may maintain a record of how many times a particular device has been changed and if the number of changes reaches or exceeds a certain threshold within a predetermined amount of time, the system may stop adjusting that device.

In some examples, a user may indirectly indicate that the system is undesirably controlling one or more devices and the system may, in response, stop controlling the device in such a manner. For example, if the system controlled a device to increase the volume of a song and the user manually reduces the volume of the device, the system may record that the user manually changed the volume setting on the device and stop actively controlling the device. Other examples of a user indirectly indicating that the system undesirably controlled one or more devices may include turning off a device and/or generally manually adjusting a device that the system changed within a predetermined amount of time. In some examples, the device may notify the system when there are manual commands entered into a device such that the system can recognize when the user is manually controlling the device.

In some embodiments, the system may provide stimuli, such as haptic feedback, informing the user whether the user is moving away or towards a targeted biometric level. In this manner, the user may learn how to consciously change their behavior such that the user may reach their desired state at process 301. For example, the user may be wearing a watch that vibrates. The system may cause the watch to vibrate in a certain manner indicating that the user is moving away from their desired state at process 301. In some examples, the watch may vibrate in a certain manner that informs the user that the user is moving towards their desired state. Some examples of different vibration profiles may include, light high frequency vibrations indicating that the user is moving towards their target state and light low frequency vibrations indication the user is moving away from their target state.

In some examples, the system may provide suggestions to the user on how the user could achieve a desired state. For example, the system may cause one of the user devices, such as smart phone 306 of FIG. 3 to suggest that the user perform some light exercise and/or drink coffee to stimulate the user into a desired state. Similarly, the system may suggest that some devices be shut off to help the user achieve a desired state. In some examples, these suggestions may be shown on a display of a user device. In some examples, the user device may provide an alert informing the user to view the recommendation. The alert may be a vibration, an audio ping, and/or a flashing LED.

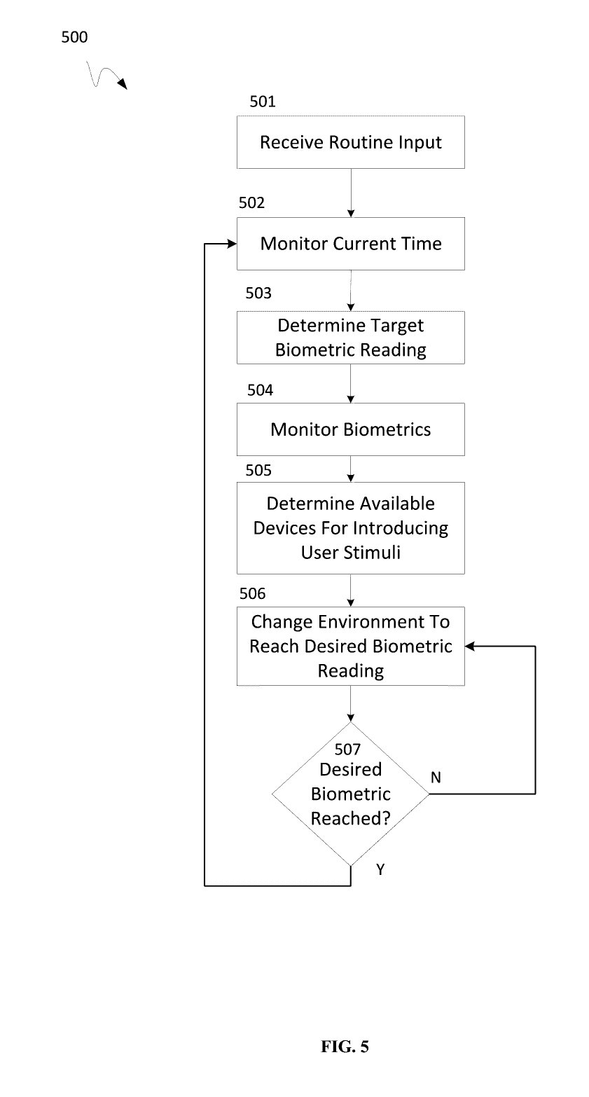

FIG. 5 illustrates exemplary method 500 for adjusting a user environment based on biometric signals to help a user maintain or change a routine. According to some embodiments, method 500 may include one or more of the processes 501-507 which may be implemented, at least in part, in the form of executable code stored on a non-transitory, tangible, machine readable media that when run on one or more processors may cause the one or more processors to perform one or more of the processes 501-507.

At process 501, the system may receive a routine input from the user. The routine may be a regiment for one or more given days. A routine may be one or more emotional, mental, and/or behavior states the user may wish to be in at a given time. Such a routine may help provide regiment to the life of the user and increase productivity. In some examples, the routine may be entered into the system by the user. An exemplary routine provided to a system may appear like the following:

| Time | State |
|---|---|
| 8:00 am | Wakeup |
| 9:01 am-10:30 am | Focus |
| 10:31 am-10:45 am | Relax |
| 10:45 am-12:00 pm | Focus |
| 12:01 pm-12:30 pm | Relax |
| 12:31 pm-3:00 pm | Focus |
| 3:01 pm-3:15 pm | Relax |
| 3:16 pm-6:00 pm | Focus |
| 6:01 pm-7:00 pm | Exercise |
| 7:01 pm-11:00 pm | Excite |
| 11:00 pm-12:00 am | Relax |
| 12:01 am | Sleep |

At process 502, the system may monitor the current time and check if it matches a time entry. For example, using the above exemplary routine, one of the times the system would compare the current time against would be 8:00 am for the wakeup state. In some embodiments, the system may have an internal clock for monitoring the current time. In some embodiments, the system may be in communications with a device that maintains the current time. When the current time matches one of the time entries for a user state, the process may continue to process 503. In some embodiments, the system may continue to process 503 at a predetermined time before a time entry.

At process 503, the system may determine target biometric readings for the user based on a state entry that is associated with the time entry of process 502. In some examples, the target biometric readings may be determined based on a prerecorded biometric signature. For example, there may be a wakeup state biometric signature, the signature may be made up of a combination of measurements such as heartbeat measurements, brainwave measurements, body heat measurements, and/or other biometric measurements. These recorded measurements may be used as the target biometric measurements for the user. An example of creating a biometric signature for a particular state is discussed in more detail below.

At process 504, the system may gather real-time and/or current biometric readings of the user from one or more devices, such as devices 103 of FIG. 1 and device 301-306 of FIG. 3. The biometric readings from the one or more devices may include several types, such as blood pressure, heart rate, brainwaves, and/or the like. The system may receive and record biometric measurements similar to process 302 of FIG. 3. The system may check if the user biometric readings match the biometric measurements for a target biometric signature determined at process 503. For example, the system at or around 8:00 am may check whether the biometric readings of the user matches the biometric signature for an awake state and/or a sleep state. If the biometric signature of the user is incongruent with the biometric signature of the target user state determined at process 503, the system may continue to process 505. For example, continuing with the 8:00 am portion of the above routine, the system would check whether the current biometric signature matches an awake state biometric signature.

At processes 505 and 506, the system may determine available devices and for introducing stimuli and change the user environment to stimulate the user in a similar manner as process 404 and 406 of FIG. 4. Continuing with the 8:00 am example, if the system determines based on the biometric readings at process 504 that the user is asleep, the system may gently attempt to bring the user to an awake state by changing the user environment. This may include controlling one or more devices to open blinds, gradually introduce audio sounds (e.g. music, audio files, etc.), brew coffee with a coffee machine, activate an aroma therapy device, gradually brighten light fixtures in a room, playing an alarm, and/or the like.

At process 507, the system may determine whether the user has reached a desired state. For example, the system may compare current/updated biometric readings with a stored biometric reading and check if the two readings match or are within a predetermined percentage of each other. In some examples, process 507 may implement a process similar to process 406 of FIG. 4. If the system determines that the current biometric readings are not sufficiently close to the target biometric readings, the system may go back to process 506 to continue adjusting the environment of the user. In some examples, the system may stop attempting to reach a target metric after a pre-determined amount of time.

Although the above illustrated example is provided for the 8:00 am entry of the exemplary routine above, processes 501-507 of method 500 also applies to the other entries. For example, the routine may include a desire to maintain a focused state at a certain time, such as when the user is working between 9:01 am-10:30 am. The system may monitor the user for biometrics indicating that the user is being distracted and control one or more devices to bring the user back to focus. For example, the system may shut off the phone of the user, play music that tends to put the user in a focused state, change the temperature in the room, or provide haptic feedback indicating to the user that they are losing focus.

As another example, the user routine may indicate that the user would like to relax between 10:31 am-10:45 am for a break. The system in response may shut off monitors, dim lights, start a videogame, play calming slow paced music, change the color of the lights in the room, and/or the like to put the user in a relaxed and/or rejuvenating state.

The routine may also include a motivated state for exercise during the times of 6:01 pm-7:00 pm. The system may check the biometric readings of the user, and if the user is in a lethargic state, attempt to control user devices to cause the user to be in a more energetic state. For example, the system may cause a user device to play upbeat motivational music to excite the user and/or play a motivational video.

In some examples, the routine may include an indication that the user would like to be in an excited and/or energetic state between 7:01 pm-11:00 pm so that the use can stay active and feel energized after a long day at work. The system may monitor the biometric readings of the user and change the user environment so that biometric readings of the user are in line with a more energized state. The system may control user devices to play party music and/or videos of nightlife activities to put the user in the mood for going out.

In some examples, the routine may include an indication that the user would like to relax to wind down between 11:00 pm-12:00 am and be ready for sleep at 12:01 am. The system may monitor the biometric readings of the user and if the user is in an energetic state, control one or more user devices to cause the user to become sleepy. This may include playing calming music and/or sounds, changing the brightness of a light source, controlling lights to be a more yellowish hue or other wavelengths of light conducive for relaxing the user, changing the temperature to one that is conducive for sleeping, and/or the like.

In some embodiments, the system may attempt to activate and control devices in an identical manner for each particular state. In this manner, the user may begin to associate changes in environment by the system for each particular state and the system may become more effective at bringing the user to a target state.

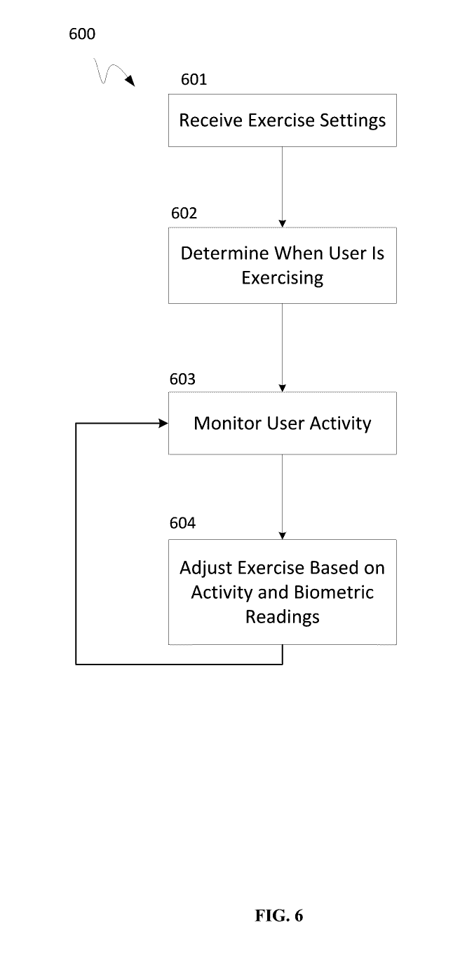

FIG. 6 illustrates an exemplary method 600 that may be implemented by a system for tailoring a fitness activity based on biometric readings. According to some embodiments, method 600 may include one or more of the processes 601-604 which may be implemented, at least in part, in the form of executable code stored on a non-transitory, tangible, machine readable media that when run on one or more processors may cause the one or more processors to perform one or more of the processes 601-604.

At process 601, the system may receive user exercise settings and/or preferences. For example, burning a certain amount of calories per day, having a certain calorie deficit, building a certain muscle group, prevent muscle atrophy, increase muscle mass and/or the like. In some examples the user may enter in physical measurements, such as height, weight, gender, age, and/or the like. This may help the system estimate how much calories were burnt by the user for a given period of time.

At process 602, the system may determine when the user is exercising and/or attempting to exercise. In some examples, the system may determine that the user is playing exercise media on a media application, a DVD player, or another media player. In some embodiments, the system may be in communication with exercise equipment and may receive indications when the equipment is turned on or is in use. In some examples, the system may check biometric readings of the user to ensure the user is the one exercising. For example, the system may check for an elevated heart rate, increased perspiration, increased muscle activity, labored breathing, motion associated with exercise, and/or other evidence of exercise.

At process 603, the system may receive biometric readings from one or more devices and use the readings to monitor the activity of the user. The system may monitor the one or more sensor devices on the body of a user throughout a period of time, such as a twenty four hour day and/or in real time. The system may monitor the number of steps the user has walked and/or run, muscle activity, heart rate, blood pressure, and/or the like. Based on the received biometric measurements, the system may estimate an activity value of the user for the time period. In some examples, the activity value may be based, at least in part, with a calories burnt estimate calculated for the user based on the biometric data.

At process 604, the system may adjust the exercise that the user is performing based on the readings collected at process 603. In some embodiments, the system may change the exercise based on the activity value. For example when an activity value indicates that the user was sedentary for a long period of time and/or for a majority of a given period of time, the system may adjust the exercise to be more rigorous. The system may set exercise equipment to a harder setting, such as increasing the speed of a run, increasing the incline of a treadmill or an elliptical machine, increasing the resistance on a stationary bike and/or rower. In some examples, the system may adjust a target distance for a run, bike, row, and/or the like based on the activity level calculated for the user.

In some examples, the system may interact with a media player and adjust the media player to adjust the exercise routine of the user. In some embodiments, an exercise media or an associated application may contain media navigation information such as chapter list, playlist, track list, timeline tags, section logs and/or the like of an exercise media (e.g. exercise video, DVD, etc.). The navigation information may be associated with information about the exercises in those sections of the media, such as muscle group categorizations (e.g. triceps, biceps, hamstring, calves, etc.), exercise types (e.g. interval training, cardio, cool down, etc.), intensity level, and/or skill levels. In some examples, the navigation information may provide information about the exercises conducted at certain portions of the media such as the length of exercises, difficulty, and/or the like. In some examples, a system may use the navigation information to control the media playing device to tailor a workout for the user. In some examples, an associated application may be backwards compatible for older DVDs. A merchant and/or DVD provider may be able to provide track lists, media navigation, and identification information for a legacy DVD. In this manner, a program application for a media player may integrate the legacy DVDs with method 600.

In some embodiments, the system may use the biometric readings collected at process 603 to predict and/or anticipate a change in exercise. For example, a user may be in the middle of a workout, but may stop for a drink of water or rest. The system may receive biometric readings at process 603 that the user has stopped exercising. This may be determined by muscle activity readings, heart beat readings, breathing rhythm readings, and/or the like. For example, the user may be watching a DVD and/or other media related to exercising a particular muscle group, such as the biceps, and the biometric readings for the that muscle group may have an abrupt stop and/or change indicating that the user has stopped exercising that muscle. In response, the system may pause the media player. In some examples the system may unpause the media player when it receives biometric readings indicating that the user is exercising that muscle again.

In some embodiments, the system may use the biometric readings collected at process 603 to determine whether the user is at a different phase or is interested in skipping to another section of a workout media. For example, the system may recognize that the media player is in the cardio portion of the workout media, but that the biometric readings of user indicates that the user is conducting or has switched to calisthenic exercises. The system, detecting this change from the biomentric readings of the user, may jump to different sections of the media, such as a particular DVD chapter, related to calisthenics.

In some embodiments, the system may change the playback speed of a workout media based on the user biometrics measured. In some examples, the system may change the playback speed to change the intensity level of a workout (e.g. faster playback for higher intensity and vice versa). The intensity level of the workout may be tailored based on the activity value calculated at process 603.

In some examples, the system may change the playback speed based on the exercise routine and/or the phase of the exercise routine. For example, if the heart rate is too high for the individual to achieve a certain goal, such as fat burning, the system may reduce the play speed of the exercise. By slowing down the exercise media, the user may lower their heart rate conducting the exercise routine at a slower pace. The system may change the playback speed to keep such that the biometric readings of the users are optimized for workout goal, such as calorie burning.

In some embodiments, the system may change the playback speed of the media based on the workout routine. For example, the user may have a workout routine that has interval training that ends with a cool down. The system may adjust the play speed of the workout media to coincide with interval training. For example, the system may cause the user to follow an interval training routine by playing workout media at a faster play speed for three minutes, then slow play speed for one minute, and then faster play speed for three more minutes. The system may also adjust the media player for when the user is in the cool down phase of their exercise routine by reducing the play speed of the media.

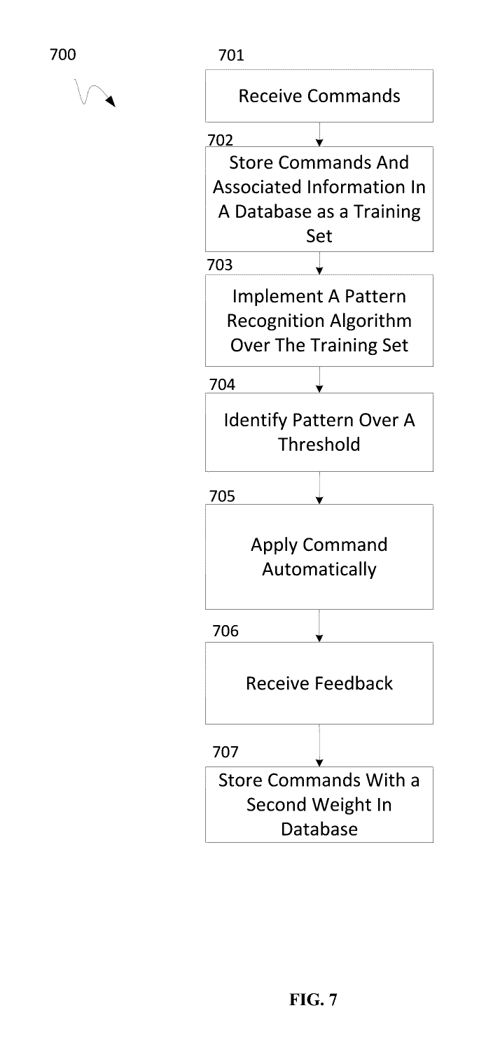

FIG. 7 illustrates an exemplary method 700 for automatically determining a target user state. In some examples, process 401 of FIG. 4 may implement method 700. According to some embodiments, method 700 may include one or more of the processes 701-707 which may be implemented, at least in part, in the form of executable code stored on a non-transitory, tangible, machine readable media that when run on one or more processors may cause the one or more processors to perform one or more of the processes 701-707.

At process 701, the system may receive a desired state command from the user for the system. In some examples the system may receive a state command from the user in a similar manner as discussed in process 401 of FIG. 4.

At process 702, the commands from process 701 may be stored in a database along with other information associated with the command, such as a user specified time (e.g. user entered date, time, day of the week), current time, user information, current biometric readings/signature, location, and/or the like to form a training set. In some examples, the state commands may be assigned a default weighting value. In some embodiments, the associated information may also be assigned a default weighting value.

At process 703, the system may run a pattern recognition algorithm over the training set. In some embodiments, the system may use one or more of the following pattern recognition algorithms and/or models for pattern recognition: clustering algorithms (e.g. k-means, hierarchical, correlation, nearest neighbor, density, etc.), classification algorithms, neural networks, Bayesian networks, regression trees, discriminant analysis, support vector machines, expectation-maximization algorithms, belief propagation algorithms, and/or the like. For example, the system may implement a clustering algorithm for a command, such as a user command setting the desired user state to a sleep state, using one or more of the associated information. The system may cluster the database for sleep state commands based on time of day, day of the week, and one or more biometric readings. As an example, the system may use k-means clustering.

At process 704, the system may identify one or more patterns in the training set and whether it crosses a certain threshold. For example, continuing with the k-means algorithm example, there may be a cluster of data points between 11 pm-12 am on Mondays for a sleep state command. The system may check if the cluster surpasses a certain density threshold, whether the cluster contains a number of data points which crosses a predetermined threshold, and/or whether the combined weighted values in the cluster passes a threshold.

At process 705, the system may automatically determine a target user state based on the one or more clusters that passed a predetermined threshold at process 704. For example, the system may set a target user state automatically based on the user command settings of the center most cluster data point of a cluster. In some examples, the system may monitor the current time, date, biometric data, and/or the like and periodically check if the current data measurements are within a particular cluster. When the current data is in one of the clusters, the system may automatically implement a target user state based on a user command associated with the cluster and/or a data point within the cluster.

At process 706, the system may receive feedback regarding the automatically implemented target user state. For example, the user may turn off and/or change the target user state within a predetermined time after the automatically implemented target user state. In some examples, the system may request input about one or more automated settings, such as whether the user liked or disliked the automatic settings.

At process 707, the feedback information at process 706 may be recorded by the system as part of the training set created at process 702. For example, if the user changes an automatically implemented target user state, the system may change the weight of the values within the cluster used to predict the target user state. In some examples, the system may ignore a related cluster once a user has turned off an automatically implemented target user state. In some examples, the system may ignore a cluster when a user has turned off an automatically implemented target user state a number of times that crosses a threshold. In some examples, if the user does not change or adjust and automatic setting, the system may increase the weight of the data points within the related cluster. In some examples, the system may add another data point to the training set with the target state. The data point for an automatically implemented target state may be given a different weighted value than a data point that is created when a user sets a user setting. The weighted value may be higher or lower than the weighted value for a manually entered user state.

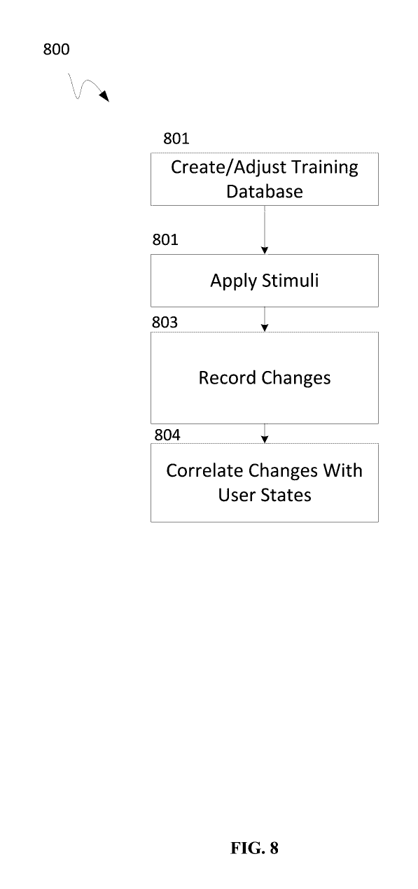

FIG. 8 illustrates an exemplary method 800 for modeling user behavioral, mental, and/or emotional states and user responses to system controlled stimuli. According to some embodiments, method 800 may include one or more of the processes 801-804 which may be implemented, at least in part, in the form of executable code stored on a non-transitory, tangible, machine readable media that when run on one or more processors may cause the one or more processors to perform one or more of the processes 801-804. In some examples, method 800 may be implements as part of process 405 of FIGS. 4 and/or 506 of FIG. 5.

At process 801, the system may create a model or a homeostasis biometric signature of the user by gathering biometric readings of the user periodically throughout a period of time. In some examples, the system may request that the user indicate when he/she is in a normal relaxed state so that the system can record biometric readings to develop a biometric model/signature for the user when in a homeostasis state. In some examples, the system may request the user to indicate a normal relaxed state several times over several days to develop a more accurate homeostasis model of the user. The system may average the recorded biometric readings to predict a homeostasis signature of the user. In some embodiments, the system may user the median, mode, a probabilistic model, and/or another mathematical algorithm designed to approximate the homeostasis signature based on multiple data points. Additionally, the system may calculate the standard deviations of each biometric reading such that the system may identify when the user is no longer in a homeostasis state.

In some examples, the system may request the user to indicate when the user is in other states, such as sleepy, focused, asleep (or soon to be), awake, exercising, tired, energized, happy, depressed, excited, and/or the like. And, the system may create models/signatures for each state of these user states in a similar manner as describe above in relation to the homeostasis user state. In some embodiments, the system may detect when the user is beyond one or more standard deviations from the homeostasis state and request input from the user about their current state. In this manner, the system may create state models and signatures of the user in a less cumbersome manner.

At process 802, the system may identify available user devices that the system may control to introduce user stimuli and control such user devices to introduce one or more stimuli. For example, they system may control a device to brighten a light, increase or decrease ambient temperature, play certain music, changing volume levels, and/or the like. The system may then check for deviations in the current biometric measurements beyond one or more standard deviations.

At process 803, the system may record and/or associate the detected biometric deviations and the amount of the deviations with the respective stimuli introduced at process 802. For example, the system may have played a particular song and found the heart rate of the user increase by 5%. The system may then store this information in a database, such as database 150 of FIG. 1, indicating that this particular song increases heart rates by 5%. In some examples the information may be stored as data points of a training set for a pattern recognition program. In some examples, the system may be creating a new training set. In some examples, the system may have an initial training set and may be updating the training set with new data points. For example, the system may have an initial training set with data points that would cause a pattern recognition program to categorize a song as calming, energetic, and/or the like. The system may be updating that training set with the data at process 802 if that song was played. As another example, the system may have training sets that would cause a pattern recognition program to recognize that dimming lights causes biometrics to move towards a depressed state. The system may have dimmed lights at process 802 and may update the training set with the information obtained at process 802,

At process 804, the stimuli may be correlated to one or more user states. In some examples, the system may determine whether deviations in biometric readings caused the user states to move closer to one or more states. For example, an excited state of a user may has a signature based on measurements of perspiration, heart rate, brainwave activity, and muscle activity. The system may determine whether, after introducing a stimulus at process 801, the biometric measurements of the user moved closer to or away from the biometric signature of a signature state. In some examples, the system may determine whether the user moved closer or farther away from a particular state by calculating and comparing the slope between a pre-stimulus state and a state signature and the slope between the post stimulus state and the state signature. As such, the system may continually update a user's profile for stimuli to apply for moving the user to particular desired states, as some stimuli that are appropriate for one user may not be appropriate for another user and stimuli that were appropriate for one user may not be appropriate for the same user at a different time.

Where applicable, various embodiments provided by the present disclosure may be implemented using hardware, software, or combinations of hardware and software. Also, where applicable, the various hardware components and/or software components set forth herein may be combined into composite components comprising software, hardware, and/or both without departing from the scope of the present disclosure. Where applicable, the various hardware components and/or software components set forth herein may be separated into sub-components comprising software, hardware, or both without departing from the scope of the present disclosure. In addition, where applicable, it is contemplated that software components may be implemented as hardware components and vice-versa.

Software, in accordance with the present disclosure, such as program code and/or data, may be stored on one or more computer readable mediums. It is also contemplated that software identified herein may be implemented using one or more general purpose or specific purpose computers and/or computer systems, networked and/or otherwise. Where applicable, the ordering of various steps described herein may be changed, omitted, combined into composite steps, and/or separated into sub-steps to provide one or more features described herein.

The foregoing disclosure is not intended to limit the present disclosure to the precise forms or particular fields of use disclosed. As such, it is contemplated that various alternate embodiments and/or modifications to the present disclosure, whether explicitly described or implied herein, are possible in light of the disclosure. Having thus described embodiments of the present disclosure, persons of ordinary skill in the art will recognize that changes may be made in form and detail without departing from the scope of the present disclosure. Thus, the present disclosure is limited only by the claims.

What is claimed is:

1. A system comprising:
one or more processors coupled to a memory that executes instructions from the memory to perform steps comprising:
determining a first state for a user;
receiving at least one biometric reading for a biometric of the user from a first user device;
determining an action for a second user device based at least in part on the at least one biometric reading and the first state for the user;
controlling the second user device to perform the action.

2. The system of claim 1, wherein the first state is characterized by at least one predetermined biometric measurement.

3. The system of claim 2, wherein determining the action for the second user device based at least in part on the at least one biometric reading and the first state of the user comprises determining that the action will cause the biometric of the user to change towards the at least one predetermined biometric measurement.

4. The system of claim 3, wherein the steps further comprises:
receiving, after controlling the second user device to perform the action, an update to the at least one biometric reading; and
controlling the second device to stop the action when a difference between the updated to the at least one biometric reading and the at least one predetermined biometric measurement is larger than a difference between the at least one biometric reading and the at least one predetermined biometric measurement.

5. The system of claim 1, the second user device comprises at least one of: a light source, media player, and an exercise machine.

6. The system of claim 1 wherein, the action comprises at least one of: changing the brightness of a light source, playing a song, changing the volume of a media player, changing chapters on a DVD, and changing the difficulty of an exercise machine.

7. A computer implemented method comprising:
determining a first state for a user;
receiving a biometric reading for a biometric of the user from a first user device;
determining a target biometric reading for the biometric of the user based on the first state;
determining an action for a second user device based at least in part on the target biometric reading and the biometric reading.

8. The method of claim 7, wherein the first state is a motivated state.

9. The method of claim 7, wherein the target biometric reading is calories burnt over a period of time.

10. The method of claim 9, wherein the available second user device is an exercise device.

11. The method of claim 10, wherein the method further comprises controlling the second user device to perform the action.

12. The method of claim 11, wherein the action is changing a difficulty of the exercise device.

13. The method of claim 8, wherein the motivated state is a biometric signature comprising a plurality of biometric values including the target biometric reading.

14. A non-transitory computer-readable medium having instructions that, when executed by a processor, performs a method comprising:
determining a first state for a user;
receiving a biometric reading for a biometric of the user from a first user device;
determining a target biometric reading based on the first state;
determining a first stimulus based on the biometric reading and the target biometric reading;
determining an available second user device capable of providing the first stimulus; and
controlling the second user device to introduce the first stimulus to the user.

15. The non-transitory computer-readable medium of claim 14, wherein the method further comprises receiving an update to the biometric reading after controlling the second user device and storing the update in a training set associated with the first stimulus.

16. The non-transitory computer-readable medium of claim 14, wherein the method further comprises receiving an update to the biometric reading after controlling the second user device and determining whether the update to the biometric reading is closer to the target biometric reading than the biometric reading.

17. The non-transitory computer-readable medium of claim 1, wherein the method further comprises controlling a third user device to introduce a second stimulus with a second device.

18. The non-transitory computer-readable medium of claim 16, wherein the method further comprises controlling a third user device to introduce a second stimulus with a second device when the update to the biometric reading is not closer to the target biometric reading.

19. The non-transitory computer-readable medium of claim 14, wherein determining the first state for the user comprises receiving a user command.

20. The non-transitory computer-readable medium of claim 14, determining the first state of for the user comprises:
receiving a plurality of user commands for the first state over a predetermined period of time;
recognizing a pattern for the plurality of user commands; and
determining the first state of the user based on the pattern.
